(19) United States
(12) Patent Application Publication
(10) Pub. No.: US 2025/0387337 A1
(43) Pub. Date: Dec. 25, 2025

(54) CONSUMER PRODUCT TABLET USING DIRECT COMPRESSION

(71) Applicant: PureLifeBiotics, LLC, Katy, TX (US)

(72) Inventors: Alexander S. PASCIAK, Katy, TX (US); Lan N. PASCIAK, Katy, TX (US)

(21) Appl. No.: 19/244,287

(22) Filed: Jun. 20, 2025

Related U.S. Application Data
(60) Provisional application No. 63/662,099, filed on Jun. 20, 2024.

(51) Int. Cl. A61K 9/20; A61K 9/00; A61K 9/28; A61K 9/46; A61K 45/06

(52) U.S. Cl. CPC A61K 9/2095; A61K 9/0007; A61K 9/007; A61K 9/2009; A61K 9/2018; A61K 9/2826; A61K 45/06

(57) ABSTRACT

A method for producing a consumer product tablet, including blending dry and/or wet phase ingredients, pressing the blended ingredients using direct compression to form a tablet, incorporating a combination of a weak acid and a weak base to produce an effervescent reaction, including a natural salt selected from the group consisting of calcium chloride and magnesium chloride to release heat upon dissolution, utilizing a water-soluble binding agent in an amount less than 10% of the tablet's weight, and forming a crystal structure on the surface of the tablet to increase the surface area.

### PRIORITY INFORMATION

This application claims the benefit of U.S. Provisional Patent Application No. 63/662,099, filed on Jun. 20, 2024, which is hereby incorporated by reference in its entirety.

### FIELD OF THE INVENTION

The example embodiments of the present invention relate to consumer product tablets (e.g., effervescent tablets) and to methods for producing such tablets.

### DISCUSSION OF THE RELATED ART

Effervescent aromatherapy shower and bath tablets have gained popularity due to their ability to enhance the sensory experience of bathing through the release of aromatic compounds. These tablets typically rely on an effervescent reaction to disperse essential oils (e.g., plant extracts) and fragrances into the air, providing a therapeutic and relaxing environment.

In the production of effervescent tablets, it is known to form granules as a pre-step for the effervescent tablet. Use of such granules does have some drawbacks. For example, the sherbets of an effervescent tablet are sensitive to humidity. In addition, the introduction of water to the sherbets releases the carbon dioxide bound in the sherbets.

Also, methods to produce effervescent tablets are known. Such a method is for example described in the European Patent Application 0 219 337. According to this method powdery dextrose or sucrose is used as a binder for the effervescent tablets. In the comparative examples of the '337 application, the binder is compared with different sugar substitutes as a binder. When using dextrose and/or sucrose as a binder, the amount of binder is 10 to 40% per weight of the whole effervescent tablet.

As described in U.S. Pat. No. 6,066,335, there is provided a method of producing effervescent tablets which consist of at least one active substance or a combination of active substances, of at least one binder, possibly of carriers as sweeteners, flavors, colorings, scents, softeners and bleaches, and of sherbets, wherein propylglycol or glycerin is used as a binder, wherein the active substance or the combination of active substances and possibly the carriers are mixed with the binder, wherein the sherbets are added to this mixture in an air-conditioned room and wherein the mixture including the sherbets is formed into tablets.

Other widely used methods of shower steamer production include wet granulation and molding. Tablet performance differs, including differences in the efficient transfer of aromatic ingredients and sealing performance.

### SUMMARY OF THE INVENTION

Accordingly, the embodiments of the present invention are directed to a variety of consumer product tablets (e.g., aromatherapy shower/bath tablet) using direct compression that substantially obviate one or more problems due to limitations and disadvantages of the related art.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, the consumer product tablet using direct compression includes a method of blending dry phase ingredients and/or wet phase ingredients, pressing the blended by direct compression, and rendering the consumer product tablet.

In another example embodiment, the consumer product tablet is made by blending dry phase ingredients and/or wet phase ingredients, pressing the blended ingredients by direct compression, and rendering the consumer product tablet.

In another example embodiment, the aromatherapy shower tablet using direct compression includes a method of blending dry phase ingredients and/or wet phase ingredients, pressing the blended by direct compression, and rendering an aromatherapy tablet.

In another example embodiment, the aromatherapy shower tablet is made by blending dry phase ingredients and/or wet phase ingredients, pressing the blended ingredients by direct compression, and rendering an aromatherapy tablet.

In the various embodiments, the aromatherapy tablet includes a combination of a weak acid and a weak base, such as a combination of citric acid and sodium bicarbonate. In the various embodiments, the aromatherapy tablet includes varying combinations of essential oils, fragrance oils, menthol, camphor, etc. In the various embodiments, the aromatherapy tablet includes a natural salt with a negative enthalpy of dissolution. In the various embodiments, the aromatherapy tablet includes calcium chloride or magnesium chloride. In the various embodiments, the aromatherapy tablet includes a crystal structure on the tablet surface (e.g., an outer surface). In the various embodiments, the aromatherapy tablet includes a crystal structure on the tablet surface (e.g., an outer surface) having peak and valley structures. In the various embodiments, the aromatherapy tablet includes Dextrose as a binding agent. In the various embodiments, the aromatherapy tablet includes a binding agent including one or more of dextrose, pregelatinized starch, hydroxypropyl methyl cellulose, polyvinyl pyrrolidone, polyethylene glycol, and the like. In the various embodiments, the aromatherapy tablet includes a binding agent that is water-soluble. In the various embodiments, the aromatherapy tablet includes a binding agent that acts as a sealant on the outer surface of the tablet.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 is a block diagram illustrating a manufacturing process for producing a consumer product tablet according to an example embodiment of the present invention; and

FIG. 2 is a flowchart illustrating a method for producing the consumer product tablet according to an example embodiment of the present invention.

### DETAILED DESCRIPTION OF THE EXAMPLE

Reference will now be made in detail to the embodiments of the present invention, examples of which are illustrated in the accompanying drawings.

Effervescent aromatherapy shower and bath tablets are a subset of consumer product tablets that have gained popularity due to their ability to enhance the sensory experience of bathing through the release of aromatic compounds. These tablets typically utilize an effervescent reaction to disperse essential oils (e.g., plant extracts) and fragrances into the air, providing a therapeutic and relaxing environment. The field of effervescent tablets encompasses a variety of applications, including personal care and household products, where the controlled release of active ingredients is desired.

The present disclosure relates to the field of consumer product tablets, with a particular focus on effervescent aromatherapy shower and bath tablets. The embodiments involve the application of direct compression techniques to streamline manufacturing processes, optimize effervescent reactions, and enhance the delivery of aromatherapy components.

Traditional methods for producing effervescent tablets often involve complex manufacturing processes, including granulation, which can be labor-intensive and costly. Granulation is a process that combines powder particles to form larger, multi-particle entities, which are then compressed into tablets. Previously, this step is required to ensure uniformity and stability of the tablet, as well as to control the rate of effervescence and release of active ingredients. However, granulation adds additional stages to the production process, increasing both time and expense.

According to the various embodiments, a method for producing effervescent aromatherapy tablets is introduced that simplifies the manufacturing process by eliminating the granulation step, thereby reducing production costs and time. Additionally, there is a need for a formulation that optimizes the effervescent reaction to enhance the release of aromatic compounds while minimizing the cooling effect that may hinder the release of aromatic compounds. The inclusion of ingredients that counteract the cooling effect and improve the distribution of aromatherapy components is described. Furthermore, a tablet design that increases surface area for more efficient transfer of aromatic ingredients, along with the use of water-soluble binders to prevent residue formation, addresses current shortcomings. A self-sealing feature to preserve volatile oils for future use is provided to further enhance the consumer experience.

In the disclosure that follows, the embodiments of the present invention are often described in connection with formulation of an effervescent aromatherapy shower/bath tablet for production (including production at mass scale) using direct compression tableting technology, but it should be readily understood that the embodiments are not so limited and may be applied to a variety of consumer product tablets.

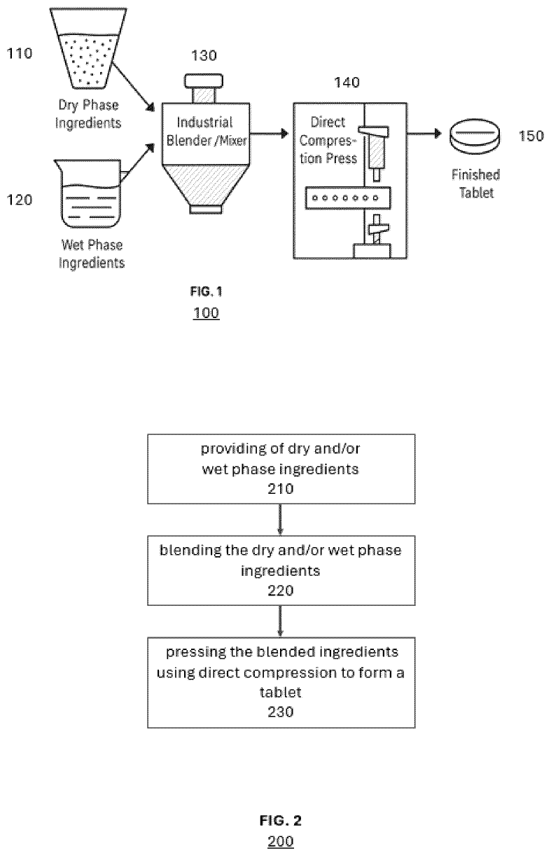

By adopting the various embodiments, manufacturing is simplified, and the costs of tableting are reduced. As compared to dry granulation and wet granulation, direct compression techniques do not require a granulator. Instead, dry phase ingredients and/or wet phase ingredients may be mixed or blended in advance of direct compression, resulting in a tablet as illustrated in FIG. 1.

FIG. 1 is a block diagram illustrating a manufacturing process 100 for producing a consumer product tablet according to an example embodiment of the present invention.

As shown in FIG. 1, the diagram illustrates a process for producing a finished tablet through a series of stages. Initially, dry phase ingredients 110 and wet phase ingredients 120 (collectively, ingredients) are introduced separately. The ingredients are then directed into an industrial blender/mixer 130, where they are combined to form a uniform mixture (e.g., homogeneous or heterogeneous). The blended mixture is subsequently transferred to a direct compression press 140, which compacts the mixture into a solid form, resulting in a finished tablet 150. This process highlights the integration of both dry and wet components to achieve the desired tablet form.

FIG. 2 is a flowchart illustrating a method 200 for producing the consumer product tablet according to an example embodiment of the present invention.

At 210, method 200 begins with the providing of dry and/or wet phase ingredients (as described in FIG. 1). At 220, the method 200 mixes/blending the dry and/or wet phase ingredients, ensuring a uniform mixture (e.g., homogeneous or heterogeneous). At 230, the method 200 presses the blended ingredients using direct compression to form a tablet (e.g., tablet 150).

The various ingredients may include, for example, a weak acid and a weak base to facilitate an effervescent reaction within the tablet; a natural salt, such as calcium chloride or magnesium chloride to release heat upon dissolution; a water-soluble binding agent, constituting less than 10% of the tablet's weight, to maintain structural integrity. A crystal structure may be formed on the tablet surface to increase surface area, enhancing dissolution properties. Additionally, a self-sealing feature may be formed to preserve volatile essential oils and fragrance oils after partial use, ensuring product longevity.

In the various embodiments, methods for production of an aromatherapy tablet using direct compression tableting technology are provided. The methods utilize direct compression technology and result in a free-flowing powder, compressible using relatively low compressive forces, and resulting in a hard tablet out of the direct compression tableting press. There are several features and advantages of the methods which result in improved aromatherapy performance when moisture is introduced (e.g., shower, bath, steam room, etc.).

First, effervescence is optimized. Effervescent tablets, which may be made using a combination of a weak acid and a weak base, such as a combination of citric acid and sodium bicarbonate, result in an endothermic reaction. Optimization of the effervescent formula in aromatherapy is critical since the aromatherapy components of the tablet, which may further include varying combinations of essential oils, fragrance oils, menthol, camphor, etc., require heat to properly transfer to the air to create an aromatherapy experience. As the effervescent reaction itself removes heat from the tablet, it is important to strike a balance between tablet temperature and CO₂ off-gassing. Another avenue is the inclusion of a natural salt with a negative enthalpy of dissolution. Calcium chloride or magnesium chloride may be used as “ice melt” because they release heat when they dissolve in water. Including these ingredients in a steamer (e.g., shower, bath, steam room, etc.) counteracts the cooling effect of the effervescent reaction and enables more efficient distribution of the aromatherapy ingredients into the air.

Second, surface area is increased. By using a slowly dissolving large grain component combined with a quickly dissolving component (e.g., sodium carbonate, sodium sesquicarbonate, etc.), once the tablet activates a crystal structure appears on the tablet surface (e.g., an outer surface) that dramatically increasing surface area by introducing peak and valley structures visible on a macro-and micro-scopic level. The increased surface area allows for high efficiency transfer of the aromatic ingredients (e.g., essential oils, fragrance oils, menthol, camphor, etc.) to the air for optimal aromatherapy experience.

Third, a water-soluble binding agent may be used. Most direct compression formulas in pharmaceuticals use microcrystalline cellulose (MCC) as a binding agent. MCC is not appropriate for an effervescent shower tablet as it will not dissolve. For example, use of MCC would leave a mess on the shower or bath floor. In the tablet formulation, dextrose may be used as the binding agent, granulated in such a way that it is suitable for direct compression. Other possible water-soluble binders include pregelatinized starch, hydroxypropyl methyl cellulose, polyvinyl Pyrrolidone, polyethylene Glycol, and the like. By using direct compression, the amount of binding agent is significantly reduced (e.g., less than 10% per weight of the whole effervescent tablet).

Fourth, a tablet that is self-sealing is provided. When a user of a shower aromatherapy tablet takes a shower, it is common that a portion of the tablet is unused when the shower is completed. Because essential oils and fragrance oils are volatile, the aromatherapy experience in a shower the following day will be reduced. Use of the dextrose binder has the added benefit of creating a “sugar coating” on the tablet surface as the tablet dries, sealing in essential oils and fragrance oils. As a result, the aromatherapy experience during the following day(s) is maintained.

Throughout this disclosure, an effervescent aromatherapy shower/bath tablet is described as an example embodiment, but the embodiments are not so limited. The embodiments may be extended to a variety of tablets including non-effervescent formulations in the personal care arena and in a variety of consumer product applications. Other examples include dry or solid bars (e.g., shampoo, conditioner, lotion, body wash, face wash, shave butter, etc.), laundry detergent tablets, dishwasher detergent tablets, etc. Yet other examples include a variety of dry or solid bars and other products that can be tableted (e.g., ingestible tablets such as supplements, food, etc. that are not effervescent tablets). Non-exhaustive additional examples include shower steamers, bath bombs (effervescent), laundry fabric softener tabs (not effervescent), liquid soap refill tablet (effervescent, add to soap pump bottle filled with water), weed killer concentrate tablet (effervescent, add to spray bottle filled with water), bug/roach killer concentrate tablet (effervescent, add to spray bottle filled with water), automotive window washer concentrate tablet (effervescent, add to your car's window washer reservoir filled with water), hydration tablet (e.g., liquid for iv intravenous (IV) delivery, or sports drink based tablet; effervescent, add to water), instant coffee tablet (effervescent, add to hot or cold water), vitamin and supplement tablet (effervescent, add to water).

It will be apparent to those skilled in the art that various modifications and variations can be made in the consumer product tablet using direct compression of the present invention without departing from the spirit or scope of the invention. Thus, it is intended that the embodiments of present invention cover the modifications and variations of this invention provided they come within the scope of the appended claims and their equivalents.

What is claimed is:

1. A method for producing a consumer product tablet, comprising:
blending dry phase ingredients and/or wet phase ingredients to produce a blended composition;
pressing the blended composition by direct compression; and
rendering the consumer product tablet.

2. The method of claim 1, wherein the consumer product tablet is an aromatherapy shower tablet.

3. The method of claim 2, wherein the aromatherapy tablet includes a combination of a weak acid and a weak base.

4. The method of claim 2, wherein the aromatherapy tablet includes a combination of citric acid and sodium bicarbonate.

5. The method of claim 2, wherein the aromatherapy tablet includes a combination of two or more of an essential oil, fragrance oil, menthol, and/or camphor.

6. The method of claim 2, wherein the aromatherapy tablet includes a natural salt with a negative enthalpy of dissolution.

7. The method of claim 2, wherein the aromatherapy tablet includes calcium chloride or magnesium chloride.

8. The method of claim 2, wherein the aromatherapy tablet includes a crystal structure on an outer surface of the aromatherapy tablet.

9. The method of claim 2, wherein the aromatherapy tablet includes a crystal structure on an outer surface of the aromatherapy tablet having peak and valley structures.

10. The method of claim 2, wherein the aromatherapy tablet includes dextrose as a binding agent.

11. The method of claim 2, wherein the aromatherapy tablet includes a binding agent that is water-soluble.

12. The method of claim 2, wherein the aromatherapy tablet includes a binding agent including one or more of dextrose, pregelatinized starch, hydroxypropyl methyl cellulose, polyvinyl pyrrolidone, and/or polyethylene glycol.

13. The method of claim 2, wherein the aromatherapy tablet includes a binding agent that acts as a sealant on an outer surface of the aromatherapy tablet.

14. A consumer product tablet comprising:
a combination of a weak acid and a weak base configured to produce an effervescent reaction upon contact with water;
a natural salt selected from the group consisting of calcium chloride and magnesium chloride, configured to release heat upon dissolution;
a water-soluble binding agent present in an amount less than 10% of the consumer product tablet's weight; and
a crystal structure on an outer surface of the consumer product tablet, the crystal structure formed by a combination of slowly and quickly dissolving components, wherein the crystal structure is configured to increase the surface area of the tablet.

15. The consumer product tablet of claim 14, wherein the weak acid is citric acid and the weak base is sodium bicarbonate.

16. The consumer product tablet of claim 14, wherein the water-soluble binding agent is selected from the group consisting of dextrose, pregelatinized starch, hydroxypropyl methyl cellulose, polyvinyl pyrrolidone, and polyethylene glycol.

17. The consumer product tablet of claim 14, further comprising a self-sealing feature configured to preserve essential oils and fragrance oils after partial use.

18. The consumer product tablet of claim 17, wherein the self-sealing feature is achieved through the formation of a sugar coating from the dextrose binder as the tablet dries.

19. A method for producing a consumer product tablet, comprising:
blending dry and/or wet phase ingredients;
pressing the blended ingredients using direct compression to form a tablet;
incorporating a combination of a weak acid and a weak base to produce an effervescent reaction;
including a natural salt selected from the group consisting of calcium chloride and magnesium chloride to release heat upon dissolution;
utilizing a water-soluble binding agent in an amount less than 10% of the tablet's weight; and
forming a crystal structure on the surface of the tablet to increase the surface area.

20. The method of claim 19, wherein the weak acid is citric acid and the weak base is sodium bicarbonate.

21. The method of claim 19, wherein the water-soluble binding agent is selected from the group consisting of dextrose, pregelatinized starch, hydroxypropyl methyl cellulose, polyvinyl pyrrolidone, and polyethylene glycol.

22. The method of claim 19, further comprising forming a self-sealing feature to preserve volatile essential oils and fragrance oils after partial use.

23. The method of claim 22, wherein the self-sealing feature is achieved through the formation of a sugar coating from the dextrose binder as the tablet dries.
